(19) United States
(12) Patent Application Publication
(10) Pub. No.: US 2021/0358574 A1
(43) Pub. Date: Nov. 18, 2021

(54) METHODS OF SCALING COMPUTATIONAL GENOMICS WITH SPECIALIZED ARCHITECTURES FOR HIGHLY PARALLELIZED COMPUTATIONS AND USES THEREOF

(71) Applicants: THE BROAD INSTITUTE, INC., Cambridge, MA (US); THE GENERAL HOSPITAL CORPORATION, Boston, MA (US); PRESIDENT AND FELLOWS OF HARVARD COLLEGE, Cambridge, MA (US)

(72) Inventors: Gad Getz, Boston, MA (US); Amaro Taylor-Weiner, Cambridge, MA (US); Francois Aguet, Cambridge, MA (US)

(73) Assignees: THE BROAD INSTITUTE, INC., Cambridge, MA (US); THE GENERAL HOSPITAL CORPORATION, Boston, MA (US); PRESIDENT AND FELLOWS OF HARVARD COLLEGE, Cambridge, MA (US)

(21) Appl. No.: 17/284,708

(22) PCT Filed: Oct. 15, 2019
(86) PCT No.: PCT/US19/56289

§ 371 Date: Apr. 12, 2021

Related U.S. Application Data
(60) Provisional application No. 62/746,547, filed on Oct. 16, 2018.

(51) Int. Cl. G16B 50/00; G16B 20/00

(52) U.S. Cl. CPC G16B 50/00; G16B 20/00

(57) ABSTRACT

The disclosure relates to methods for increasing the speed and efficiency of computational genomics. In particular, the disclosure relates to methods of scaling computational genomics by using one or more specialized architectures for highly parallelized computations, such as graphics processing units (GPUs), tensor processing units (TPUs), and field programmable gate arrays (FPGAs), and the like, to compute the computational genomics calculations.

### STATEMENT REGARDING FEDERALLY SPONSORED RESEARCH

This invention was made with government support under Grant No. 268201000029C, awarded by the National Institutes of Health. The government has certain rights in the invention.

### FIELD OF THE DISCLOSURE

The disclosure relates to methods for increasing the speed and efficiency of computational genomics. More particularly, the disclosure relates to methods of scaling computational genomics with one or more specialized architectures for highly parallelized computations, such as, for example, graphics processing units (GPUs), tensor processing units (TPUs), field programmable gate arrays (FPGAs), and other such architectures.

### BACKGROUND OF THE DISCLOSURE

Current genomics methods and pipelines were designed to handle tens to thousands of samples, but will need to scale to millions of samples to match the pace of data and hypothesis generation in biomedical science. Unfortunately, the computational costs associated with processing such large datasets is prohibitive with current central processing unit (CPU) based methodologies. For example, current methods in population genetics, such as genome-wide association studies (GWAS) or mapping of quantitative trait loci (QTLs), involve billions of regressions between genotypes and phenotypes, and such large-scale datasets are typically analyzed on large-scale clusters of CPUs that contain hundreds or thousands of cores. Unfortunately, such large-scale clusters are very expensive, and still take a significant amount of CPU time to run computational genomics analyses. Accordingly, there is an urgent need for methods that increase the speed and efficiency of computational genomics.

### SUMMARY OF THE DISCLOSURE

The present disclosure is based, at least in part, on the discovery that computational genomics algorithms/computations may be run on specialized architectures for highly parallelized computations, such as graphics processing units (GPUs), tensor processing units (TPUs), field programmable gate arrays (FPGAs), and the like, at speeds that are orders of magnitude faster than similar computations conducted on central processing unit (CPU) based systems. Additionally, implementations on such specialized architectures (e.g., GPUs, TPUs, FPGAs, etc.) are scalable. The availability of recent libraries such as PyTorch and TensorFlow has also contributed to the accelerations in computational genomics algorithms/computations observed herein.

In one aspect, the disclosure provides a method for conducting computational genomics analyses, including the steps of receiving, at a capable node in a computer network, a large-scale biologic dataset; storing the dataset in a central processor unit (CPU) memory; passing the dataset, or a selection thereof, to one or more specialized architecture for highly parallelized computations; computing, at the specialized architecture, the computational genomics analysis; and outputting one or more results of the computational genomics analysis.

In an embodiment, the large-scale biologic dataset includes bulk sequence data (e.g., genomic sequence data, variant sequence data, transcriptome sequence data) or bulk gene expression data. In another embodiment, the large-scale biologic dataset includes large-scale molecular measurements of biological material from bulk tissue and/or single cells. Optionally, the large-scale molecular measurements of biological material from bulk tissue and/or single cells includes measurements of the epigenome, measurements of the proteome, measurements of the metabolome and/or measurements of the microbiome (among other forms of data/measurements contemplated).

In an embodiment, the one or more specialized architectures for highly parallelized computations include a graphics processor unit (GPU), a tensor processing unit (TPU), and/or a field programmable gate array (FPGA).

In an embodiment, the computational analysis is a genotype association test, optionally the computational analysis is selected from the group consisting of a genome wide association study (GWAS), a quantitative trait loci (QTL) analysis, and a Bayesian non-negative matrix factorization. In an embodiment, the dataset is a variant call format (VCF) dataset. In an embodiment, the dataset is a count matrix.

In an embodiment, the computing further includes the steps of: identifying, at the specialized architecture, genotypes, phenotypes, and/or covariates; correcting, at the specialized architecture, for covariates; computing, at the specialized architecture, an association calculation; and computing, at the specialized architecture, a P-value correction.

In an embodiment, an algorithm initially designed for CPU computing is executed by placing computationally intensive operations on the specialized architecture for highly parallelized computations. Optionally, the computationally intensive operations include linear algebra operations, matrix operations and/or vector calculus operations, optionally including matrix multiplication, matrix inversion and/or gradient computations (among others contemplated).

In an embodiment, the method of computing further includes the steps of computing, at the specialized architecture, a W matrix containing signature or cluster activations; computing, at the specialized architecture, an H matrix containing patient/sample signature or cluster memberships; and computing, at the specialized architecture, a lambda term which regularizes W and H.

In one aspect, the disclosure provides an apparatus, including: one or more network interfaces to communicate with a computer network; a central processor unit (CPU) coupled to the network interfaces and adapted to execute one or more processes; a CPU memory configured to store a process executable by the CPU, one or more specialized architecture for highly parallelized computations coupled to the network interfaces and adapted to execute one or more processes; a specialized architecture memory configured to store a process executable by the specialized architecture, the process when executed operable to: receive, at a capable node in a computer network, a large-scale biologic dataset; store the dataset in the CPU memory; pass the dataset, or a selection thereof, to the one or more specialized architecture; compute, at the specialized architecture, the computational genomics analysis; and output one or more results of the computational genomics analysis.

In an embodiment, the computational analysis is selected from the group consisting of a genome wide association study (GWAS), a quantitative trait loci (QTL) analysis, and a Bayesian non-negative matrix factorization.

In an embodiment, the dataset is a variant call format (VCF) dataset. In an embodiment, the dataset is a count matrix.

In an embodiment, the computing further includes the ability to: identify, at the specialized architecture, genotypes, phenotypes, and/or covariates; correct, at the specialized architecture, for covariates; compute, at the specialized architecture, an association calculation; and compute, at the specialized architecture, a P-value correction.

In an embodiment, the method of computing further includes the steps of conducting an NMF analysis according to blocks 231-234 in FIG. 3A.

In an embodiment, the method of computing includes the steps of conducting an analysis of a bulk dataset according to blocks 300-340 in FIG. 3B.

In one aspect, the disclosure provides a tangible, non-transitory, computer-readable media having software encoded thereon, the software, when executed by a processor on a particular device, operable to: receive, at a capable node in a computer network, a large-scale biologic dataset; store the dataset in a central processor unit (CPU) memory; pass the dataset, or a selection thereof, to one or more specialized architecture for highly parallelized computations; compute, at the specialized architecture, the computational genomics analysis; and output one or more results of the computational genomics analysis.

### DEFINITIONS

Unless defined otherwise, all technical and scientific terms used herein have the meaning commonly understood by a person skilled in the art to which this disclosure belongs. The following references provide one of skill with a general definition of many of the terms used in this disclosure: The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term about.

By “biologic sample” is meant any tissue, cell, fluid, or other material derived from an organism or collected from the environment.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Where applicable or not specifically disclaimed, any one of the embodiments described herein are contemplated to be able to combine with any other one or more embodiments, even though the embodiments are described under different aspects of the disclosure. These and other embodiments are disclosed and/or encompassed by, the following Detailed Description.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 shows a comparison of central processing unit (CPU) and graphics processing unit (GPU) architecture.

FIG. 2 is a schematic block diagram depicting a GWAS analysis as implemented on a GPU according to an exemplary embodiment of the disclosure.

FIG. 3A is a schematic block diagram depicting an exemplary Bayesian non-negative matrix factorization (NMF).

FIG. 3B is a generic schematic block diagram depicting an exemplary process flow of the instant disclosure, as implemented upon a GPU.

FIGS. 4A-4B are process flow diagrams illustrating exemplary computations for a GWAS in TensorFlow showing input-output operations color coded to show operations taking place on the CPU relative to those occurring on the GPU. FIG. 4A shows the GPU computation graph. FIG. 4B shows the input and output processing which occurs on CPU.

FIGS. 5A-5G demonstrate that the disclosed graphics processing unit (GPU) implementations resulted in dramatically increased performance for quantitative trait loci (QTL) and signature analysis. FIG. 5A shows a graph demonstrating average run time to compute 10,000 iterations of Bayes NMF using SignatureAnalyzer (SA) in R (left bar) and SignatureAnalyzer-GPU (SA-GPU; right bar). FIG. 5B shows a correlation heat map of mutation signatures derived from the R and GPU implementations of SignatureAnalyzer using the same input mutation counts matrix. FIG. 5C shows t-Distributed Stochastic Neighbor Embedding (t-SNE) of 1 million embryonic mouse brain cells. Shading indicates clustering based on SA-GPU decomposition performed in ˜15 minutes. FIG. 5D shows a comparison of runtimes for cis-QTL (FastQTL on CPU; left-hand bars and tensorQTL on GPU; right-hand bars) and trans-QTL (tensorQTL on CPU and GPU). FIG. 5E is a graph showing that TensorQTL benchmarking based on a random data set representing up to 50,000 people, each with 10⁷ genotypes of common variants, could be performed at a scope that was orders of magnitude beyond current QTL studies while also involving 20-fold more associations than the largest genome wide association study (GWAS) performed to date (lines show GPU runtime of tensorQTL for the indicated numbers of samples and phenotypes). FIG. 5F is a graph showing the reproducibility of the disclosed GPU implementations based on a Matrix eQTL analysis. FIG. 5G is a graph showing that empirical cis-eQTL p values from the V7 GTEx release replicated using tensorQTL. Error bars indicate standard deviation of the mean.

FIG. 6 demonstrates the performance of matrix multiplication on a single CPU core (2.30 GHz Intel Xeon) vs. a GPU (Nvidia Tesla P100), using NumPy (compiled with OpenBLAS) and PyTorch. Runtimes were measured for multiplication of two random (uniform ˜U[0,1]) square matrices (in 32-bit floating point) with the indicated dimensions. For the ‘PyTorch GPU’ runtimes, only the matrix multiplication itself was timed. For the ‘PyTorch GPU w/copy’ runtimes, the copy of the two input matrices from CPU to GPU memory was included in the timing. Runtimes are shown as the median and median absolute deviation of 15 iterations each.

FIGS. 7A-7C show performance scaling of SignatureAnalyzer-GPU (the Bayesian non-negative matrix factorization of the instant disclosure). FIG. 7A shows that SignatureAnalyzer-GPU runtime scaled linearly as a function of the number of samples. FIG. 7B shows the observed cumulative runtime for 20 runs of SignatureAnalyzer-GPU on a virtual machine configured with one or two GPUs (Nvidia Tesla V100). FIG. 7C shows the average number of signatures detected with one or two GPUs, which indicated that the results were equivalent. The PCAWG mutation counts matrix was used for all comparisons. Error bars represent standard deviation.

FIGS. 8A-8C show observed GPU performance scaling of tensorQTL. FIG. 8A demonstrates the observed GPU-to-CPU runtime ratio for tensorQTL, across the indicated phenotype and sample sizes, for 10⁷ common variants. The ratio was observed as non-constant due to data load and CPU-to-GPU memory input/output times (“i/o”) that were more limiting for large sample sizes (number of individuals). FIG. 8B shows observed CPU runtime of tensorQTL for the indicated range of sample and phenotype sizes (left panel). CPU runtimes scaled linearly, as was demonstrated by the collapse of the compute time when measured as a function of number of operations (approximated as phenotypes×samples×variants; middle panel), whereas GPU runtimes did not show this collapse for large sample sizes due to i/o limitations (right panel). FIG. 8C shows nominal significant trans-eQTL p values from the V6p GTEx release replicated using tensorQTL.

### DETAILED DESCRIPTION OF THE DISCLOSURE

The present disclosure is based, at least in part, on the discovery that computational genomics algorithms/computations may be run on specialized architectures for highly parallelized computations, such as graphics processing units (GPUs), tensor processing units (TPUs), field programmable gate arrays (FPGAs), and the like, at speeds that are orders of magnitude faster than similar computations conducted on central processing unit (CPU) based systems. Additionally, implementations that employ specialized architectures for highly parallelized computations are scalable.

### Overview

Current genomics methods and pipelines have been designed to handle tens to thousands of samples, but will soon need to scale to millions to match the pace of data and hypothesis generation in biomedical science. Due to the continuing decrease in sequencing costs and growth of large-scale genomic projects, datasets are reaching sizes of millions of samples or single cells. The need for increased computational resources, most notably runtime, to process these growing datasets will become prohibitive without improving the computational efficiency and scalability of methods. For example, methods in population genetics, such as genome-wide association studies (GWAS) or mapping of quantitative trait loci (QTL), involve billions of regressions between genotypes and phenotypes. Currently, the state-of-the-art infrastructures for performing these tasks are large-scale clusters of central processing units (CPUs), often with thousands of cores, resulting in significant costs¹ (960 cores on a standard Google Cloud machine currently costs $7,660.80 per day of compute). In contrast to CPUs, a single graphics processing unit (GPU) contains thousands of cores at a much lower price per core (Nvidia's P100 has 3,584 cores and currently costs $35.04 per day of compute).

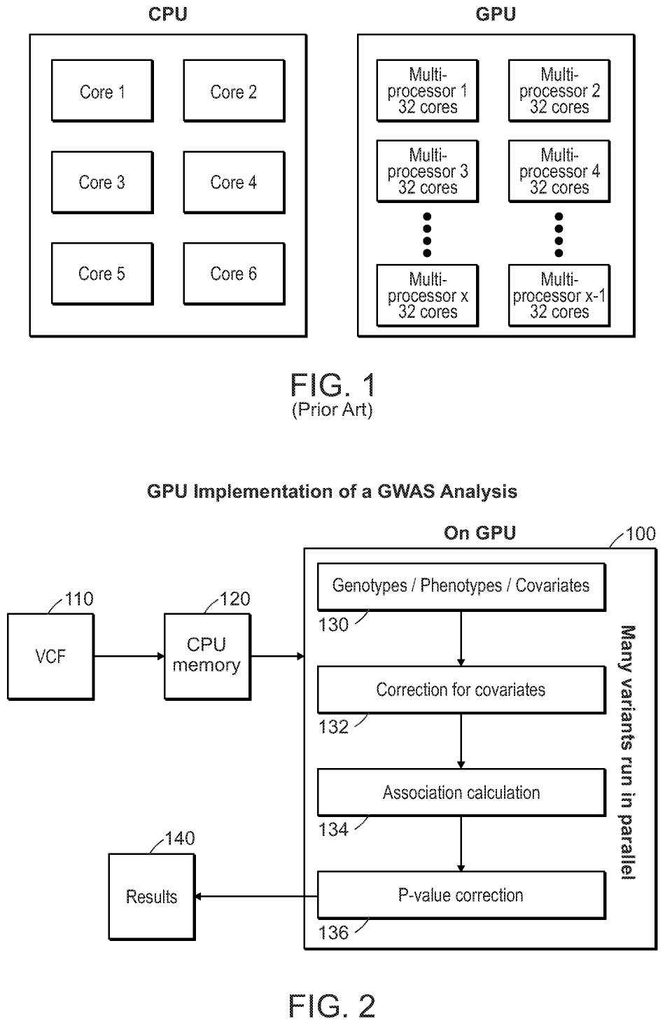

In contrast to CPUs, graphics processing units (GPUs) are programmable logic chips (e.g., processors) that are specialized for display functions (e.g., output to a monitor). For example, a typical GPU renders images, video, etc., for computers monitor, and are typically located on plug-in cards, but may also be located in a chipset on the motherboard or in the same chip as the CPU. As shown in FIG. 1, CPUs feature relatively few cores (e.g., Intel's i9 CPU has 6 cores), whereas a single GPU contains hundreds or thousands of cores (e.g. Nvidia's P100 GPU has 3,584 cores). GPUs are optimized for computing highly parallelizable mathematical operations, and use of GPUs has revolutionized the field of machine learning, where training extremely deep artificial neural networks requires performing billions of identical operations. A GPU typically performs parallel operations on multiple sets of data, and may be used as a vector processor for non-graphics applications. However, while there have been attempts made using specialized software to analyze large-scale datasets on GPUs, the speedups realized in such attempts were far more modest than those that have now been demonstrated herein, using the specialized architecture(s) and processes disclosed herein. In addition, the range of applications identified herein for use of specialized architectures for highly parallelized computations for analysis of large-scale biologic datasets, such as genomic datasets, is dramatically expanded herein, as compared to such past attempts.

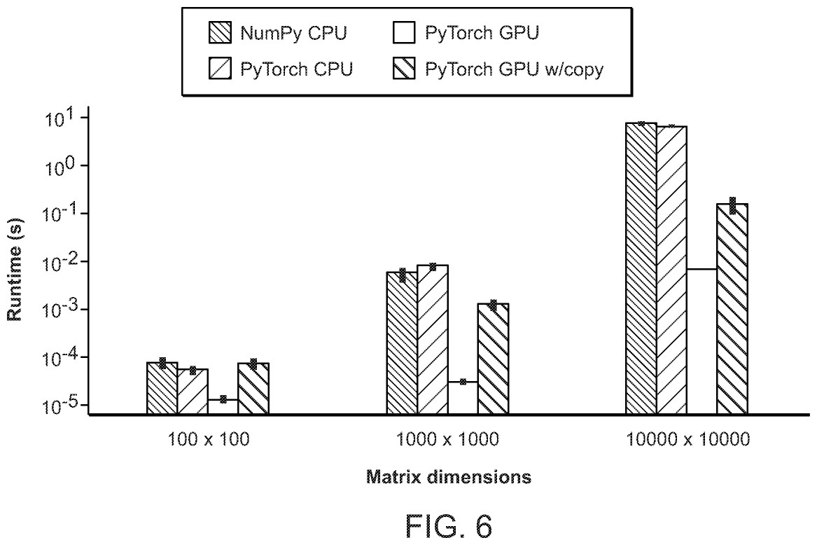

Previous work has already demonstrated the benefits of using GPUs to scale bioinformatics methods²⁻⁶. However, these implementations were often complex and based on specialized libraries, limiting their extensibility and adoption. In contrast, recent open-source libraries such as TensorFlow⁷ or PyTorch⁸, which were developed for machine learning applications but implement general-purpose mathematical primitives and methods (e.g., matrix multiplication), make development of GPU-compatible tools widely accessible to the research community (TensorFlow and PyTorch are noted as convenient libraries for computing on specialized architectures for highly parallelized computations, such as GPUs, etc., and it is contemplated that more generally any library such as TensorFlow and PyTorch can be used with the instant disclosure). These libraries offer several major advantages: (i) they implement most of the functionalities of CPU-based scientific computing libraries such as NumPy and thus are easy to use for implementing various algorithms; (ii) they easily handle data transfer from the computer's memory to the GPU's internal memory, including in batches, and thus greatly facilitate computations on large datasets (e.g., large genotype matrices) that do not fit into the GPU's memory; (iii) they are trivial to install and run, enabling easy sharing of methods; and (iv) they can run seamlessly on both CPUs and GPUs, permitting users without access to GPUs to test and use them, without loss of performance compared with other CPU-based implementations (FIG. 6). Moreover, users do not need to explicitly specify how to parallelize algorithms across the GPU cores. It was therefore reasoned that the use of these libraries might result in significant improvements in computational efficiency, thereby enabling scaling of computational genomics methods to millions of samples.

It has now been demonstrated herein that high efficiency at low cost could be achieved by leveraging general-purpose libraries for GPU computing, such as PyTorch and TensorFlow. Greater than 200-fold decreases in runtime were demonstrated, at about 5-10-fold reductions in cost relative to CPUs. It is contemplated that the accessibility of these libraries will lead to wide-spread adoption of GPUs in computational genomics.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

As described herein, computational genomics may be implemented on specialized architectures for highly parallelized computations (e.g., GPUs, TPUs, FPGAs, etc.) and may result in significant improvements to analytical efficiency and scalability when applied to hundreds of thousands of patient samples. Furthermore, existing frameworks for specialized architecture computation may be used to facilitate such analyses. In particular, the specialized architecture-based techniques herein may be implemented on computational genomics problems involving continuous variables, discrete variables, Bayesian non-negative matrix factorization (NMF and Bayesian NMF), and the like.

FIG. 2 is a schematic block diagram depicting an exemplary genotype association analysis as implemented on GPU 100. For example, initial data set 110 (e.g., in variant call format (VCF)) may be input to CPU memory 120, and then parsed to GPU 100, where an exemplary processing flow includes identifying genotypes/phenotypes/covariates 130, correcting for covariates 132, conducting the Association calculation 134, making a P-value correction, to yield the results 140.

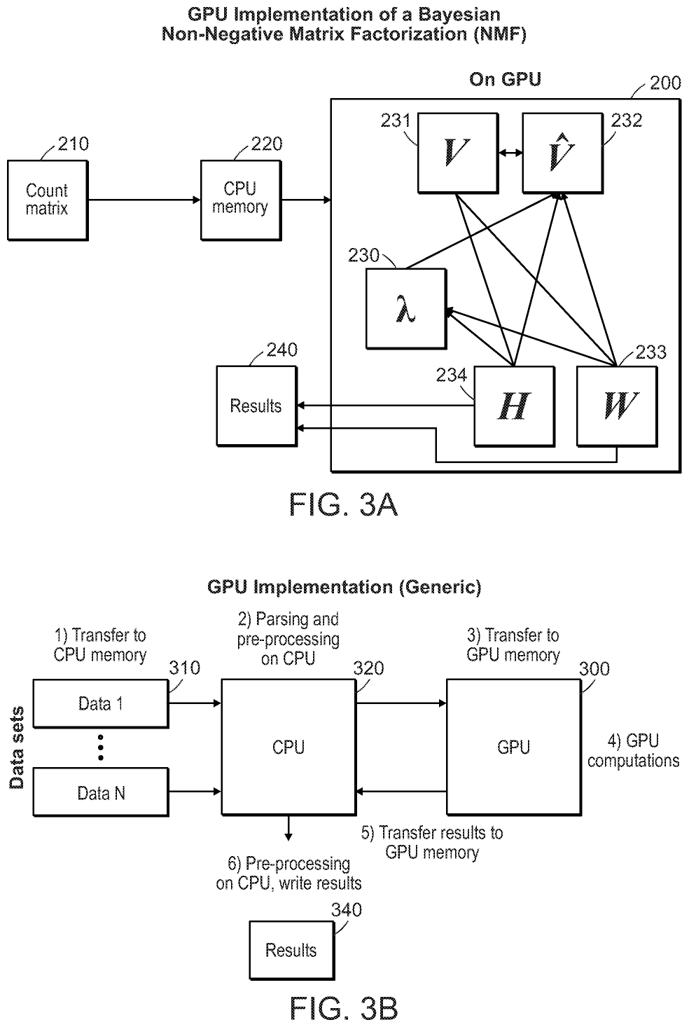

FIG. 3A is a schematic block diagram depicting an exemplary Bayesian non-negative matrix factorization (NMF) as implemented on GPU 200. For example, initial data set 210 (e.g., a count matrix) may be input to CPU memory 220, and then parsed to GPU 200, where an exemplary factorization includes factoring λ 230, V 231, \tilde{V} 232, W 233, and H 234, to yield the results 240. Optimization was performed upon H and W to minimize the distance between V and \tilde{V} and the lambda regularization term. This was performed in an iterative procedure where H and W were optimized according to update rules described in Kim et al.⁵

FIG. 3B is a generalized schematic block diagram depicting an exemplary workflow as implemented on GPU 200. For example, initial data set 310 may be input to CPU memory 320, and then passed (in whole or in part) to GPU 300, where highly parallelized computations are performed, to yield the results 340.

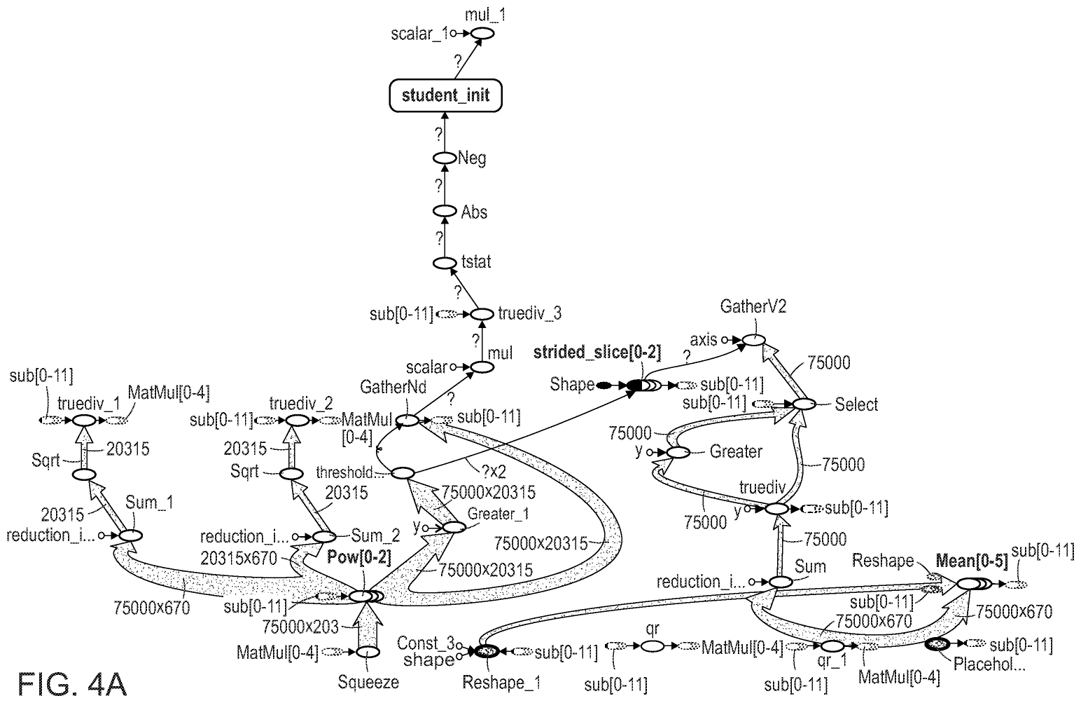

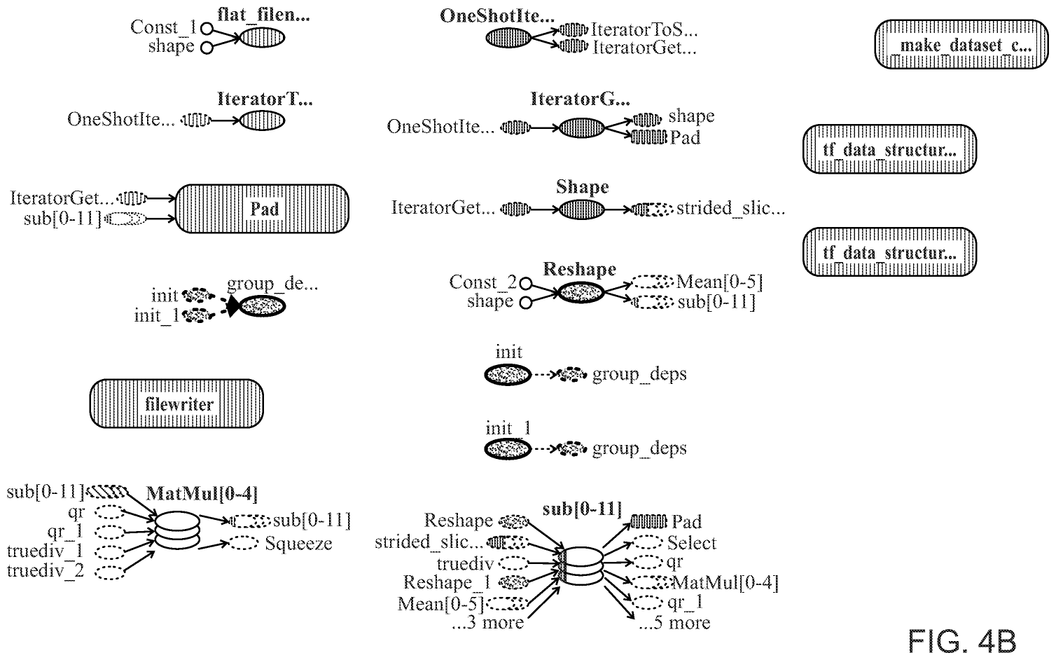

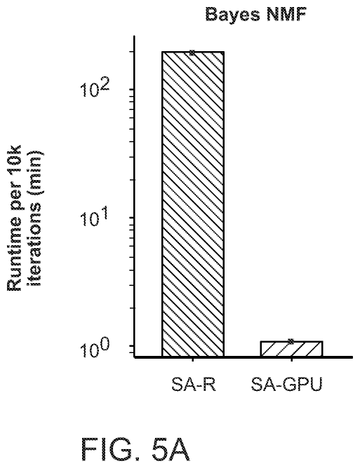

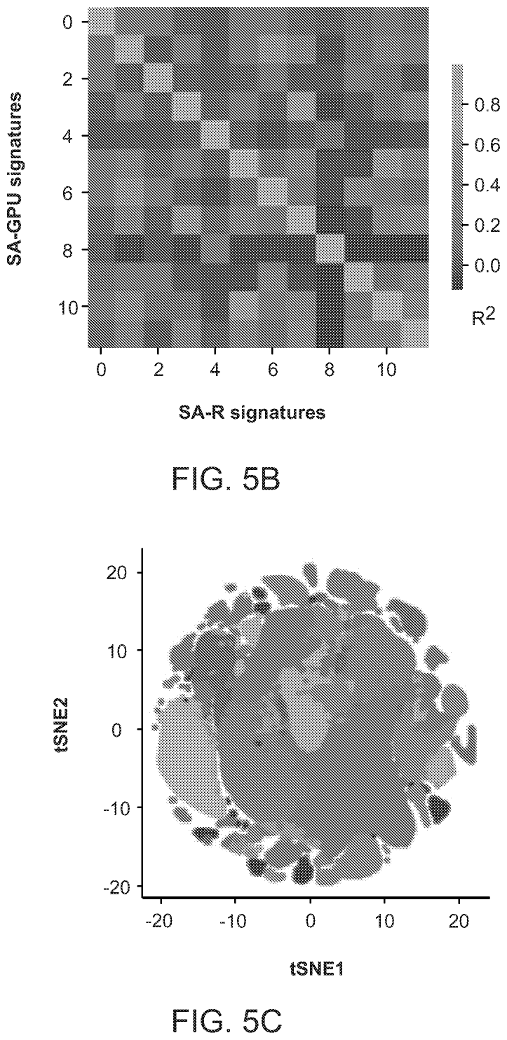

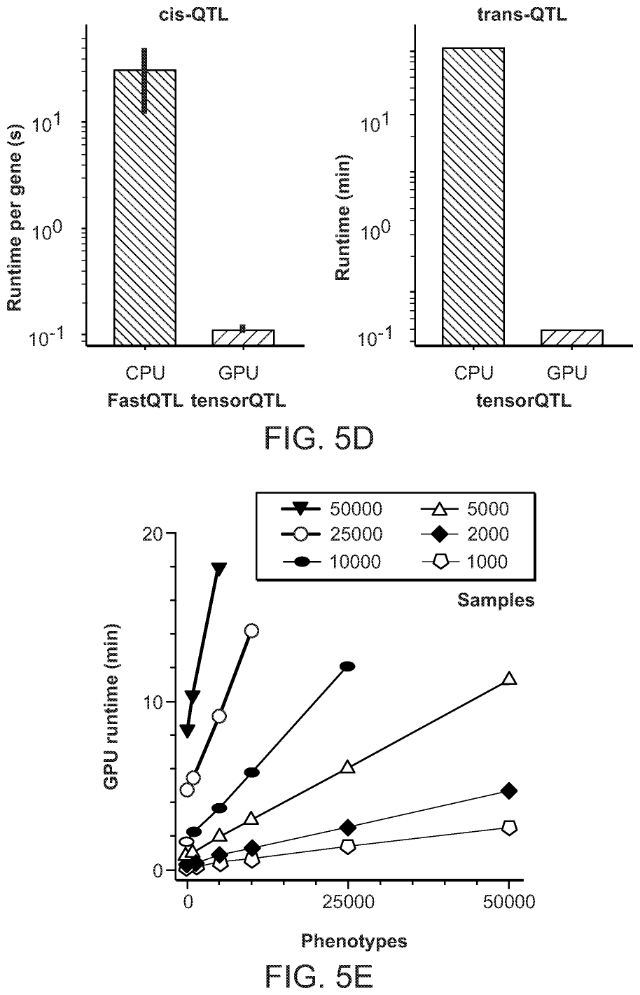

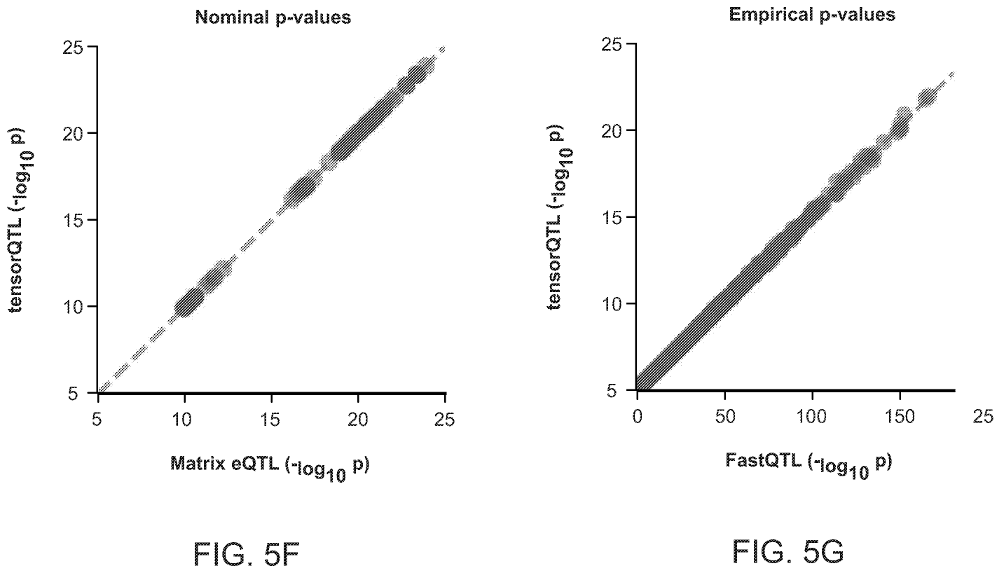

FIGS. 4A-4B are process flow diagrams illustrating exemplary computations for a genotype association study in TensorFlow showing input-output operations color coded to show operations taking place on the CPU (Blue) relative to those occurring on the GPU (Green). FIG. 5A highlights the mathematical function calls computed to generate association p-values and corrected p-values. FIG. 5D shows the entire Tensorflow computation graph including input/output operations which occur on CPU.

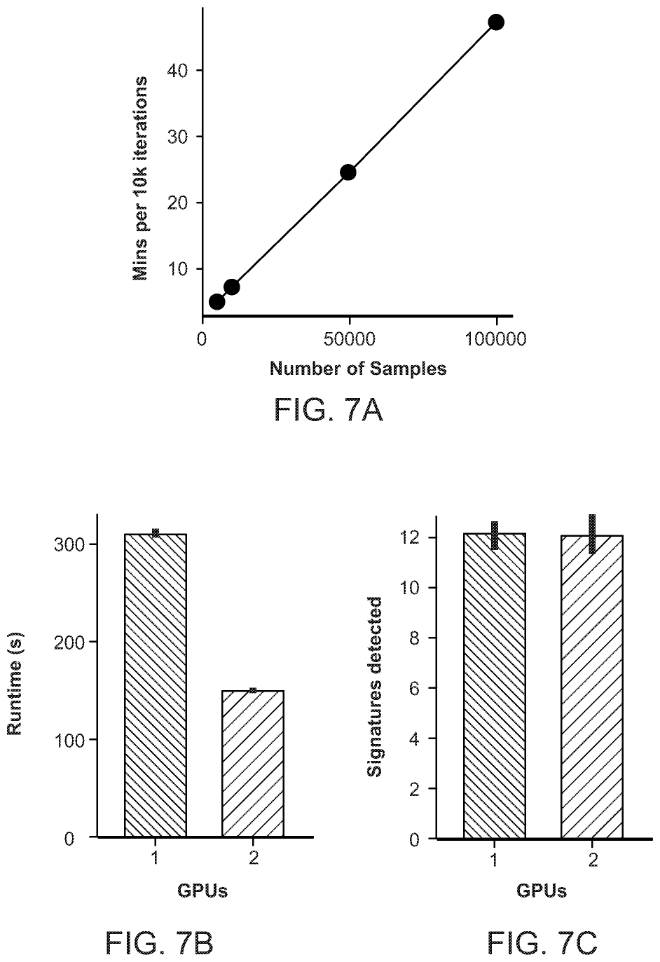

To examine the efficiency and benchmark the use of TensorFlow and PyTorch for large-scale genomic analyses on GPUs, methods for two commonly performed computational genomics tasks were re-implemented: (i) QTL mapping^{9,10} (termed ‘tensorQTL’ herein¹¹) and Bayesian non-negative matrix factorization¹² (named SignatureAnalyzer-GPU herein¹³). The same scripts were executed in identical environments (configured with and without a GPU) and they were also compared to previous CPU-based implementations. As a baseline, the performance of individual mathematical operations such as matrix multiplication was also benchmarked, for which up to 1000-fold faster runtimes on a GPU was observed vs. a single CPU core (FIG. 6 and data not shown). For SignatureAnalyzer-GPU (SA-GPU)¹³, the mutation counts matrix generated by the Pan-Cancer Analysis of Whole Genomes (PCAWG) Consortium, which contains 2,624 tumors represented by 1,697 mutational features of somatic single nucleotide variants as well as short insertions and deletions (defined based on their sequence contexts)¹⁴, was used. Unexpectedly, the instant PyTorch implementation ran approximately 200 times faster on a GPU than the current implementation of SignatureAnalyzer (SA) in R (run on a single CPU core), with mean times for 10,000 iterations of 1.09 minutes using SA-GPU vs. 194.8 minutes using SA (FIG. 5A). Using simulated data, it was demonstrated that SA-GPU scaled linearly with the number of samples (FIG. 7A). When applied to previously published mutational signatures generated by SA¹⁵, the results of the two methods were observed to be essentially identical, taking into account the stochastic nature of the underlying algorithm (mean R²=0.994, min R²=0.960; FIG. 7B). Additionally, the performance of SA-GPU on multiple GPUs was tested, a task that was easily achieved in PyTorch and enabled, for example, faster hyperparameter optimization. For 20 decompositions using the same data as above, it was observed that performance scaled linearly with the number of GPUs and yielded equivalent results (FIGS. 7B and 7C).

To further demonstrate the scalability of Bayesian non-negative matrix factorization to millions of data points, SA-GPU was used to identify cell types and their associated transcriptional programs from single-cell RNA sequencing of 1 million mouse brain cells (SRA: SRP096558, FIG. 5C). The average time per SA-GPU run was 14.5 minutes (using a V100 Nvidia GPU; average over 10 runs) corresponding to an average of 6,853 matrix updates per run. A similar analysis on a CPU would require >2 days per run. The instant analysis was able to identify 32 distinct transcriptional programs (FIG. 5C).

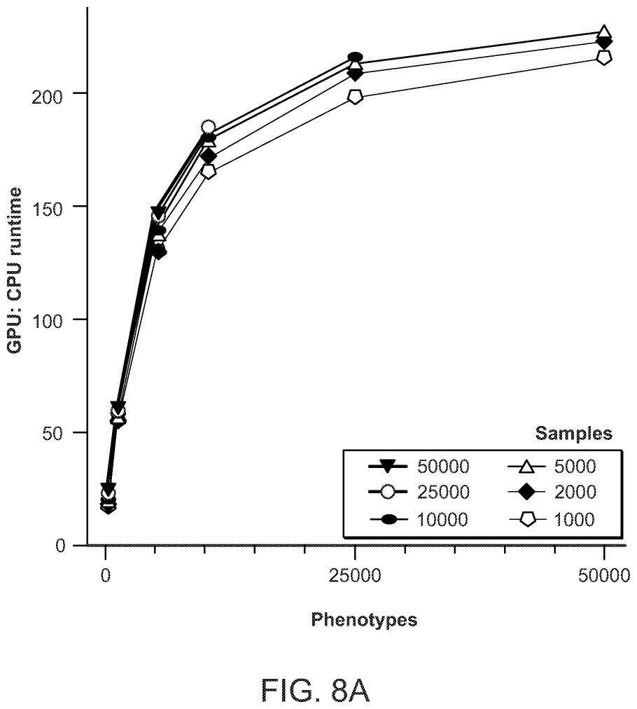

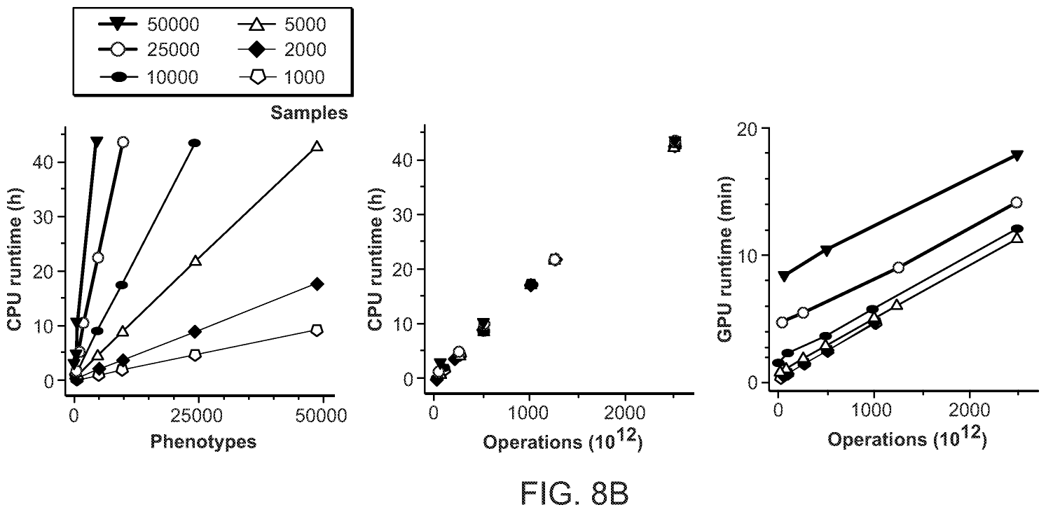

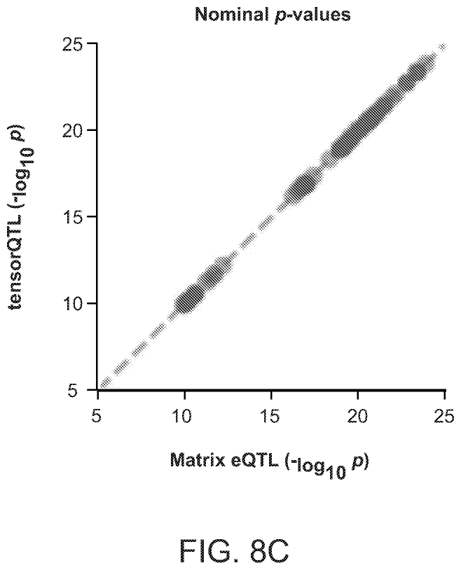

For tensorQTL¹¹ benchmarking, random data were generated representing up to 50,000 people, each with 10⁷ genotypes representing common variants. For each individual, up to 50,000 phenotypes (e.g., gene expression values) were also simulated, resulting in 500×10⁹ all-against-all association tests (each calculated for up to 50,000 individuals). Notably, as shown in FIG. 5E, this analysis was orders of magnitude beyond the scope of current QTL studies and involved 20-fold more associations than the largest GWAS performed so far¹. The instant implementation of cis-QTL mapping with permutations to estimate the empirical false discovery rate was >250 times faster than the current state-of-the-art implementation (FastQTL¹⁰; FIG. 5D). Likewise, trans-QTL mapping (i.e., 500 billion regressions) took less than 10 minutes, a ˜200× increase in speed compared to running on a CPU (FIGS. 1C and 8A). The current implementation does not scale linearly as a function of samples (FIG. 8B) due to limitations in data transfer from the memory of the CPU to the GPU, rather than computational capacity; this additional optimization has been left for future work (FIGS. 5E and 8B). Data from the V6p and V7 releases of GTEx¹⁶ generated using Matrix eQTL⁹ and FastQTL¹⁰, respectively, were used to demonstrate the reproducibility of the instant implementation (FIGS. 5G and 8C).

In addition to the savings in computation time, implementation in TensorFlow or PyTorch also results in significant cost savings—at the time of the instant disclosure, GPU compute time costs ˜$0.50-0.75/hour on multiple cloud platforms, as compared to ˜$0.01-0.05/hour for a CPU core. Thus, the same analyses were ˜5-10 fold cheaper on GPUs.

In summary, implementation of many commonly used methods in genomics based on new GPU-compatible libraries has been identified as a means to vastly increase runtime and reduce costs compared to CPU-based approaches. Indeed, by simply re-implementing current methods, an order-of-magnitude higher increase in speed was realized herein than may be achieved through sophisticated approximations for optimizing runtimes on CPUs^{17,18}. The instant findings indicate that the scale of computations made possible with specialized architectures for highly parallelized computations, such as GPUs, TPUs, and FPGAs (among other such architectures), will enable investigation of previously unanswerable hypotheses involving more complex models, larger datasets, and more accurate empirical measurements. For example, the GPU implementation has enabled computation of empirical p values for trans-QTL, which is cost-prohibitive on CPUs. Similarly, the instant results have demonstrated that GPU-based approaches (as well as TPU-based, FPGA-based, etc. approaches) enable scaling of single-cell analysis methods to millions of cells. Given the availability of libraries that obviate the need for specialized GPU programming, a transition to GPU-based computing is now contemplated for a wide range of computational genomics methods.

### Methods

tensorQTL

The core of tensorQTL is a reimplementation of FastQTL¹⁰ in TensorFlow⁷ and relies on pandas_plink (github.com/limix/pandas-plink) to efficiently read genotypes stored in PLINK¹⁹ format into dask arrays²⁰.

The following QTL mapping modalities are implemented:
cis-QTL: nominal associations between all variant-phenotype pairs within a specified window (default: ±1 Mb) around the phenotype (transcription start site for genes), as implemented in FastQTL.
cis-QTL: beta-approximated empirical p values, based on permutations of each phenotype, as implemented in FastQTL.
cis-QTL: beta-approximated empirical p values for grouped phenotypes; for example, multiple splicing phenotypes for each gene, as implemented in FastQTL.
conditionally independent cis-QTL, following the stepwise regression approach described in GTEx¹⁶.
interaction QTLs: nominal associations for a linear model that includes a genotype×interaction term.
trans-QTL: nominal associations between all variant-phenotype pairs. To reduce output size, only associations below a given p value threshold (default: 1e-5) are stored.
trans-QTL: beta-approximated empirical p values for inverse-normal-transformed phenotypes, in which case the genome-wide associations with permutations of each phenotype are identical. To avoid potentially confounding cis effects, the computation is performed for each chromosome, using variants on all other chromosomes.

### Benchmarking

To benchmark tensorQTL, its trans-QTL mapping performance on a machine with and without an attached GPU was compared, and cis-QTL mapping relative to the CPU-based FastQTL¹⁰ (an optimized QTL mapper written in C++) was also examined. For FastQTL, the runtime per gene was computed by specifying the gene and cis-window using the—include-phenotypes and—region options, respectively. The cis-mapping comparisons were performed using skeletal muscle data from the V6p release of GTEx¹⁶. To facilitate the comparison of GPU vs. CPU performance when mapping trans-QTLs across a wide range of sample sizes, randomly generated genotype, phenotype, and covariate matrices were used. All tensorQTL benchmarks were conducted on a virtual machine on Google Cloud Platform with 8 Intel Xeon CPU cores (2.30 GHz), 52 GB of memory, and a Nvidia Tesla P100 GPU. For CPU-based comparisons, computations were limited to a single core.

### SignatureAnalyzer-GPU

SA-GPU is a PyTorch reimplementation of SignatureAnalyzer²¹, a method for identification of somatic mutational signatures using Bayesian NMF²². SignatureAnalyzer was originally developed in R and is available for download at software.broadinstitute.org/cancer/cga/. Currently, SA-GPU requires the input data matrix and decomposition matrices (W and H) to fit into GPU memory; however, since high-memory GPUs are readily available (e.g., Nvidia Tesla v100 has 16 GB), this should not limit its practical use. In case data sizes were to exceed this limit, the method is easily extensible to multiple GPUs using shared memory with built-in PyTorch methods.

SA-GPU can run a single Bayesian NMF or an array of decompositions in parallel, leveraging multiple GPUs. Users should specify a data likelihood function (Poisson or Gaussian) and either exponential or half-normal prior distributions on the elements of W and H, corresponding to L1 or L2 regularization, respectively.

### Benchmarking

To benchmark the performance of SA-GPU, SA-GPU was compared with the previous implementation in R. The R implementation was run using R 3.2.3 with the ‘Matrix’ package for efficient matrix operations. All SA-GPU benchmarks were conducted on a virtual machine on Google Cloud Platform with 12 Intel Xeon CPU cores (2.30 GHz), 20 GB of memory, and a Nvidia Tesla V100 GPU. For CPU-based comparisons, a single core was used.

Availability of data and materials: All software is available on Github and implemented in Python using open source libraries.

tensorQTL is released under the open-source BSD 3-Clause License and is available at: github.com/broadinstitute/tensorQTL¹¹.

SignatureAnalyzer-GPU is released under the open-source BSD 3-Clause License and is available at: github.com/broadinstitute/SignatureAnalyzer-GPU¹³.

The foregoing description has been directed to specific embodiments; however, it will be apparent to the skilled artisan that other variations and modifications may be made to the described embodiments, while continuing to provide some or all of the above-described advantages. For instance, it is expressly contemplated that the components and/or elements described herein can be implemented as an apparatus that comprises at least one network interface that communicates with a communication network, a processor (e.g., a CPU, GPU, and the like) coupled to the at least one network interface, and a memory configured to store program instructions executable by the processor. It is also expressly contemplated that in addition to GPUs, similar specialized architectures, including some that already exist, such as tensor processing units (TPUs), or others that may become available in the future, could provide a comparable benefit to GPUs or could even improve implementation of the instant methods further. Further, it is expressly contemplated that the components and/or elements described herein can be implemented as software being stored on a tangible (non-transitory) computer-readable medium (e.g., disks, CDs, RAM, EEPROM, and the like) having program instructions executing on a computer, hardware, firmware, or a combination thereof. Accordingly, this description is to be taken only by way of example and not to otherwise limit the scope of the embodiments herein. Therefore, it is the object of the appended claims to cover all such variations and modifications as come within the true spirit and scope of the embodiments herein.

### REFERENCES

1. Bycroft C, Freeman C, Petkova D, Band G, Elliott L T, Sharp K, et al. The UK Biobank resource with deep phenotyping and genomic data. Nature [Internet]. Nature Publishing Group; 2018 [cited 2018 Oct. 12]; 562:203-9. Available from: www.nature.com/articles/s41586-018-0579-z.

2. McArt D G, Bankhead P, Dunne P D, Salto-Tellez M, Hamilton P, Zhang S-D. cudaMap: a GPU accelerated program for gene expression connectivity mapping. BMC Bioinformatics [Internet]. BioMed Central; 2013 [cited 2018 Oct. 18]; 14:305. Available from: bmcbioinformatics.biomedcentral.com/articles/10.1186/1471-2105-14-305.

3. Mejia-Roa E, Tabas-Madrid D, Setoain J, Garcia C, Tirado F, Pascual-Montano A. NMF-mGPU: non-negative matrix factorization on multi-GPU systems. BMC Bioinformatics [Internet]. BioMed Central; 2015 [cited 2018 Oct. 18]; 16:43. Available from: bmcbioinformatics.biomedcentral.com/articles/10.1186/s12859-015-0485-4.

4. Schatz M C, Trapnell C, Delcher A L, Varshney A. High-throughput sequence alignment using Graphics Processing Units. BMC Bioinformatics [Internet]. BioMed Central; 2007 [cited 2018 Oct. 18]; 8:474. Available from: bmcbioinformatics.biomedcentral.com/articles/10.1186/1471-2105-8-474.

5. Nobile M S, Cazzaniga P, Tangherloni A, Besozzi D. Graphics processing units in bioinformatics, computational biology and systems biology. Brief Bioinform [Internet]. Narnia; 2016 [cited 2019 May 20]; 18:bbw058. Available from: academic.oup.com/bib/article-lookup/doi/10.1093/bib/bbw058.

6. Angermueller C, Parnamaa T, Parts L, Stegle O. Deep learning for computational biology. Mol Syst Biol [Internet]. EMBO Press; 2016 [cited 2019 May 20]; 12:878. Available from: msb.embopress.org/content/12/7/878.

7. Abadi M, Agarwal A, Barham P, Brevdo E, Chen Z, Citro C, et al. TensorFlow: Large-Scale Machine Learning on Heterogeneous Distributed Systems [Internet]. 2016. Available from: arxiv.org/abs/1603.04467.

8. Paszke A, Gross S, Chintala S, Chanan G, Yang E, DeVito Z, et al. Automatic differentiation in PyTorch. 2017.

9. Shabalin A A. Matrix eQTL: ultra fast eQTL analysis via large matrix operations. Bioinformatics [Internet]. Oxford University Press; 2012 [cited 2018 Oct. 1]; 28:1353-8. Available from: www.ncbi.nlm.nih.gov/pubmed/22492648.

10. Ongen H, Buil A, Brown A A, Dermitzakis E T, Delaneau O. Fast and efficient QTL mapper for thousands of molecular phenotypes. Bioinformatics [Internet]. 2016 [cited 2018 Oct. 1]; 32:1479-85. Available from: www.ncbi.nlm.nih.gov/pubmed/26708335.

11. Aguet F, Taylor-Weiner A. tensorqtl. Github. github.com/broadinstitute/tensorqtl (2019).

12. Kim J, Mouw K W, Polak P, Braunstein L Z, Kamburov A, Kwiatkowski D J, et al. Somatic ERCC2 mutations are associated with a distinct genomic signature in urothelial tumors. Nat Genet [Internet]. NIH Public Access; 2016 [cited 2018 Aug. 23]; 48:600-6. Available from: www.ncbi.nlm.nih.gov/pubmed/27111033.

13. Taylor-Weiner A, Aguet F. Signature Analyzer-GPU. Github. github.com/broadinstitute/SignatureAnalyzer-GPU/(2019).

14. Alexandrov L, Kim J, Haradhvala N J, Huang M N, Ng A W T, Boot A, et al. The Repertoire of Mutational Signatures in Human Cancer. bioRxiv [Internet]. Cold Spring Harbor Laboratory; 2018 [cited 2018 Oct. 1]; 322859. Available from: www.biorxiv.org/content/early/2018/05/15/322859.

15. Haradhvala N J, Kim J, Maruvka Y E, Polak P, Rosebrock D, Livitz D, et al. Distinct mutational signatures characterize concurrent loss of polymerase proofreading and mismatch repair. Nat Commun [Internet]. Nature Publishing Group; 2018 [cited 2018 Aug. 23]; 9:1746. Available from: www.nature.com/articles/s41467-018-04002-4.

16. GTEx Consortium. Genetic effects on gene expression across human tissues. Nature [Internet]. Nature Publishing Group; 2017 [cited 2018 Oct. 1]; 550:204-13. Available from: www.nature.com/doifinder/10.1038/nature24277.

17. Loh P-R, Kichaev G, Gazal S, Schoech A P, Price A L. Mixed-model association for biobank-scale datasets. Nat Genet [Internet]. Nature Publishing Group; 2018 [cited 2019 Feb. 7]; 50:906-8. Available from: www.nature.com/articles/s41588-018-0144-6.

18. Zhou W, Nielsen J B, Fritsche L G, Dey R, Gabrielsen M E, Wolford B N, et al. Efficiently controlling for case-control imbalance and sample relatedness in large-scale genetic association studies. Nat Genet [Internet]. Nature Publishing Group; 2018 [cited 2019 Feb. 7]; 50:1335-41. Available from: www.nature.com/articles/s41588-018-0184-y.

19. Chang C C, Chow C C, Tellier L C, Vattikuti S, Purcell S M, Lee J J. Second-generation PLINK: rising to the challenge of larger and richer datasets. Gigascience [Internet]. 2015 [cited 2019 May 20]; 4:7. Available from: www.ncbi.nlm.nih.gov/pubmed/25722852.

20. Rocklin M. Dask: Parallel Computation with Blocked algorithms and Task Scheduling. Proc 14th Python Sci Conf [Internet]. 2015 [cited 2019 May 20]. p. 126-32. Available from: conference.scipy.org/proceedings/scipy2015/matthew_rocklin.html.

21. Kim J, Mouw K W, Polak P, Braunstein L Z, Kamburov A, Kwiatkowski D J, et al. Somatic ERCC2 mutations are associated with a distinct genomic signature in urothelial tumors. Nat Genet [Internet]. 2016 [cited 2017 Sep. 11]; 48:600-6. Available from: www.ncbi.nlm.nih.gov/pubmed/27111033.

22. Tan V Y F, Edric C′, Evotte F′. Automatic Relevance Determination in Nonnegative Matrix Factorization with the β-Divergence [Internet]. 2012. Available from: arxiv.org/pdf/1111.6085.pdf.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

### EQUIVALENTS

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A method for conducting computational genomics analyses, comprising:
receiving, at a capable node in a computer network, a large-scale biologic dataset;
storing the dataset in a central processor unit (CPU) memory;
passing the dataset, or a selection thereof, to one or more specialized architecture for highly parallelized computations;
computing, at the specialized architecture, the computational genomics analysis; and
outputting one or more results of the computational genomics analysis.

2. The method of claim 1, wherein the large-scale biologic dataset is selected from the group consisting of bulk sequence data and bulk gene expression data, optionally wherein the bulk sequence data comprises genomic sequence data, variant sequence data and/or transcriptome sequence data.

3. The method of claim 1, wherein the large-scale biologic dataset is selected from the group consisting of large-scale molecular measurements of biological material from bulk tissue, large-scale molecular measurements of biological material from single cells, and combinations thereof, optionally wherein the large-scale molecular measurements of biological material comprise measurements selected from the group consisting of measurements of the epigenome, measurements of the proteome, measurements of the metabolome, measurements of the microbiome, and combinations thereof.

4. The method of claim 1, wherein the one or more specialized architecture is selected from the group consisting of a graphics processor unit (GPU), a tensor processing unit (TPU), a field programmable gate array (FPGA), and a combination thereof.

5. The method of claim 1, wherein the computational analysis is a genotype association test, optionally wherein the computational analysis is selected from the group consisting of a genome wide association study (GWAS), a quantitative trait loci (QTL) analysis, and a Bayesian non-negative matrix factorization.

6. The method of claim 1, wherein the dataset is a variant call format (VCF) dataset.

7. The method of claim 1, wherein the dataset is a count matrix.

8. The method of claim 1, wherein computing further comprises:
identifying, at the specialized architecture, genotypes, phenotypes, and/or covariates;
correcting, at the specialized architecture, for covariates;
computing, at the specialized architecture, an association calculation; and
computing, at the specialized architecture, a P-value correction.

9. The method of claim 1, wherein an algorithm initially designed for CPU computing is executed by placing computationally intensive operations on the specialized architecture, optionally wherein the computationally intensive operations comprise operations selected from the group consisting of linear algebra operations, matrix operations, vector calculus operations, and combinations thereof, optionally wherein the operations comprise operations selected from the group consisting of matrix multiplication, matrix inversion, gradient computations, and combinations thereof.

10. The method of claim 1, wherein computing further comprises:
computing, at the specialized architecture, a W matrix containing signature or cluster activations;
computing, at the specialized architecture, an H matrix containing patient/sample signature or cluster memberships; and
computing, at the specialized architecture, a lambda term, wherein the lambda term regularizes W and H.

11. An apparatus, comprising:
one or more network interfaces to communicate with a computer network;
a central processor unit (CPU) coupled to the network interfaces and adapted to execute one or more processes;
a CPU memory configured to store a process executable by the CPU,
one or more specialized architecture for highly parallelized computations coupled to the network interfaces and adapted to execute one or more processes;
a specialized architecture memory configured to store a process executable by the specialized architecture, the process when executed operable to:
receive, at a capable node in a computer network, a large-scale biologic dataset;
store the dataset in the CPU memory;
pass the dataset, or a selection thereof, to the one or more specialized architecture;
compute, at the specialized architecture, the computational genomics analysis; and
output one or more results of the computational genomics analysis.

12. The apparatus of claim 11, wherein the computational analysis is selected from the group consisting of a genome wide association study (GWAS), a quantitative trait loci (QTL) analysis, and a Bayesian non-negative matrix factorization.

13. The apparatus of claim 11, wherein the dataset is a variant call format (VCF) dataset.

14. The apparatus of claim 11, wherein the dataset is a count matrix.

15. The apparatus of claim 11, wherein compute further comprises:
identify, at the specialized architecture, genotypes, phenotypes, and/or covariates;
correct, at the specialized architecture, for covariates;
compute, at the specialized architecture, an association calculation; and
compute, at the specialized architecture, a P-value correction.

16. The apparatus of claim 11, wherein compute further comprises:
computing, at the specialized architecture, a W matrix containing signature or cluster activations;
computing, at the specialized architecture, an H matrix containing patient/sample signature or cluster memberships; and
computing, at the specialized architecture, a lambda term, wherein the lambda termregularizes W and H.

17. A tangible, non-transitory, computer-readable media having software encoded thereon, the software, when executed by a processor on a particular device, operable to:
receive, at a capable node in a computer network, a large-scale biologic dataset;
store the dataset in a central processor unit (CPU) memory;
pass the dataset, or a selection thereof, to one or more specialized architecture for highly parallelized computations;
compute, at the specialized architecture, the computational genomics analysis; and
output one or more results of the computational genomics analysis.
